(19) United States
(12) Patent Application Publication
(10) Pub. No.: US 2018/0296447 A1
(43) Pub. Date: Oct. 18, 2018

(54) BIOCIDAL COMPOSITIONS AND RELATED METHODS

(71) Applicant: Imerys Filtration Minerals, Inc., San Jose, CA (US)

(72) Inventor: Bo WANG, Union City, CA (US)

(21) Appl. No.: 15/569,440

(22) PCT Filed: Apr. 25, 2016
(86) PCT No.: PCT/US2016/029133

§ 371 Date: Oct. 26, 2017

Related U.S. Application Data
(60) Provisional application No. 62/153,768, filed on Apr. 28, 2015.

(51) Int. Cl. A61K 8/02; A61K 8/27; A61K 8/25; A61Q 11/00; A61Q 17/00

(52) U.S. Cl. CPC A61K 8/0245; A61K 8/27; A61K 8/25; A61Q 11/00; A61K 2800/28; A61K 2800/412; A61K 2800/61; A61K 2800/651; A61Q 17/005

(57) ABSTRACT

A biocidal composition may include perlite and a biocidal metal compound associated with the perlite. The perlite may have a top particle size (d₅₀) less than 50 μm and a median particle size (d₅₀) less than 30 μm, and the perlite may range from 0.1 percent to 20 percent by weight of the composition. A dentifrice composition may include perlite and a biocidal metal compound associated with the perlite. The perlite may have a top particle size (d₅₀) less than 50 μm, a median particle size (d₅₀) less than 30 μm, and exhibit an RDA value less than 220. The composition may include a toothpaste base. A method of making a biocidal composition may include providing perlite and contacting the perlite with a biocidal metal compound to form biocidal metal-treated perlite.

### CLAIM OF PRIORITY

This PCT International Application claims the benefit of priority of U.S. Provisional Application No. 62/153,768, filed Apr. 28, 2016, the subject matter of which is incorporated herein by reference in its entirety.

### FIELD OF THE DISCLOSURE

The present disclosure relates to biocidal compositions and related methods. In particular, the present disclosure relates to biocidal compositions including perlite and a biocidal metal compound and related methods.

### BACKGROUND OF THE DISCLOSURE

Abrasive compositions may have a variety of desirable uses. For example, abrasive compositions may be used for cleaning and/or polishing various surfaces. For example, abrasive compositions may be used to clean and/or polish teeth. Such dentifrice compositions may include pastes or powders for cleaning and/or polishing teeth. Toothpaste is a commonly known example of a dentifrice composition, which typically has a paste-like form and which may include one or more components, such as, for example, binders, humectants, abrasives, detergents, and flavoring agents. The abrasive component in toothpaste serves to improve its cleaning effectiveness. However, while abrasives may improve cleaning effectiveness, they may also lead to undesirable erosion of the teeth. In addition, although abrasives may improve the effectiveness of cleaning teeth, abrasives may not be effective at reducing undesirable germs and bacteria associated with oral hygiene.

One composition that has been added to toothpaste to improve its cleaning effectiveness is perlite. Perlite is an example of a naturally-occurring glass, such as, for example, an amorphous volcanic glass having a relatively high water content. By virtue of its relatively high water content, perlite expands when heated, for example above about 850-900′C. While perlite may improve the cleaning effectiveness of toothpaste, it may also lead to premature degradation of teeth due to its inherently abrasive nature. Furthermore, perlite may not be effective at reducing undesirable germs and bacteria associated with oral hygiene (e.g., Streptococcus mutans).

Thus, it may be desirable to provide compositions that assist with the effectiveness of cleaning teeth, but which do not lead to premature erosion of the teeth due to excessive abrasiveness. It may also be desirable to provide compositions that assist with reducing or killing undesirable germs and bacteria.

### SUMMARY

In the following description, certain aspects and embodiments will become evident. It should be understood that the aspects and embodiments, in their broadest sense, could be practiced without having one or more features of these aspects and embodiments. Thus, it should be understood that these aspects and embodiments are merely exemplary.

One aspect of the disclosure relates to a biocidal composition including perlite and a biocidal metal compound associated with the perlite. The perlite may have a top particle size (d₉₀) less than 50 μm and a median particle size (d₅₀) less than 30 μm. The perlite may range from 0.1 percent to 20 percent by weight of the composition.

According to a further aspect, a dentifrice composition may include perlite and a biocidal metal compound associated with the perlite. The perlite may have a top particle size (d₉₀) less than 50 μm, a median particle size (d₅₀) less than 30 μm, and the dentifrice composition may exhibit a relative dentin abrasion (RDA) value less than 200. RDA testing is a method of measuring of the erosive effect on tooth dentin of abrasives in compositions for cleaning teeth, and RDA value is standardized in accordance with DIN/ISO standard 11609, a standard that has been adopted by the American Dental Association (ADA). Higher RDA values indicate higher levels of abrasiveness.

According to another aspect, the dentifrice composition may include a toothpaste base. For example, the toothpaste base may include at least one of binders, humectants, abrasives, detergents, and flavoring agents.

According to a further aspect, a method of making a biocidal composition may include providing perlite and contacting the perlite with a biocidal metal compound to form biocidal metal-treated perlite. Providing the perlite may include providing perlite having a top particle size (d₉₀) less than 50 μm and a median particle size (d₅₀) less than 30 μm, and the perlite may range from 0.1 percent to 20 percent by weight of the composition.

Additional objects and advantages of the disclosure will be set forth in part in the description which follows, or may be learned by practice of the disclosed embodiments.

Aside from the arrangements set forth above, the embodiments could include a number of other arrangements, such as those explained hereinafter. It is to be understood that both the foregoing description and the following description are exemplary only.

### BRIEF DESCRIPTION OF THE DRAWINGS

The accompanying drawings, which are incorporated in and constitute a part of this description, illustrate several exemplary embodiments and together with the description, serve to explain the principles of the embodiments. In the drawings,

FIG. 1 is a scanning electron micrograph of Harborlite® 2000.

FIG. 2 is a scanning electron micrograph of the fine fraction of the a classified, expanded perlite sample of Example 1.

FIG. 3 is a graph showing Relative Dentin Abrasion (RDA) test results for three examples of natural glass vs. top particle size (d₉₀).

FIG. 4 is a graph showing Pellicle Cleaning Ratio (PCR) test results for three examples of natural glass vs. top particle size (d₉₀).

FIG. 5 is a scanning electron micrograph of Example 4 at ×20,000 magnification.

FIG. 6 is a scanning electron micrograph of Example 4 at ×90,000 magnification.

### DESCRIPTION OF EXEMPLARY EMBODIMENTS

Reference will now be made in more detail to a number of exemplary embodiments of the invention.

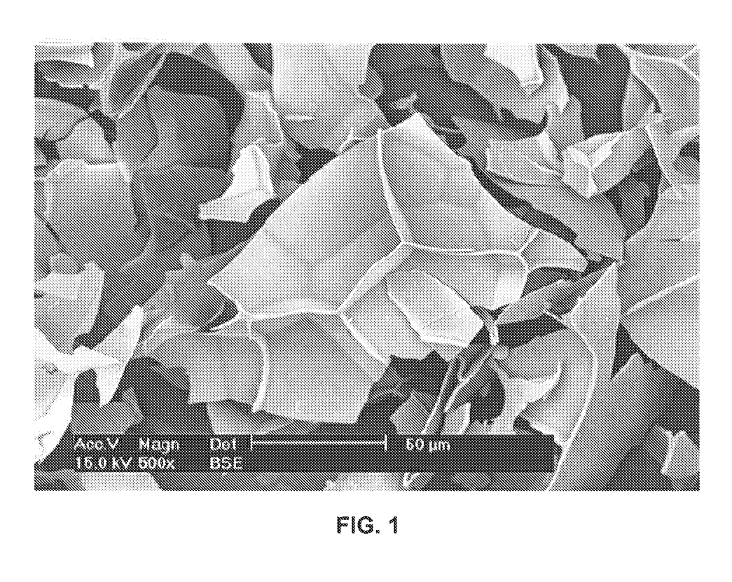

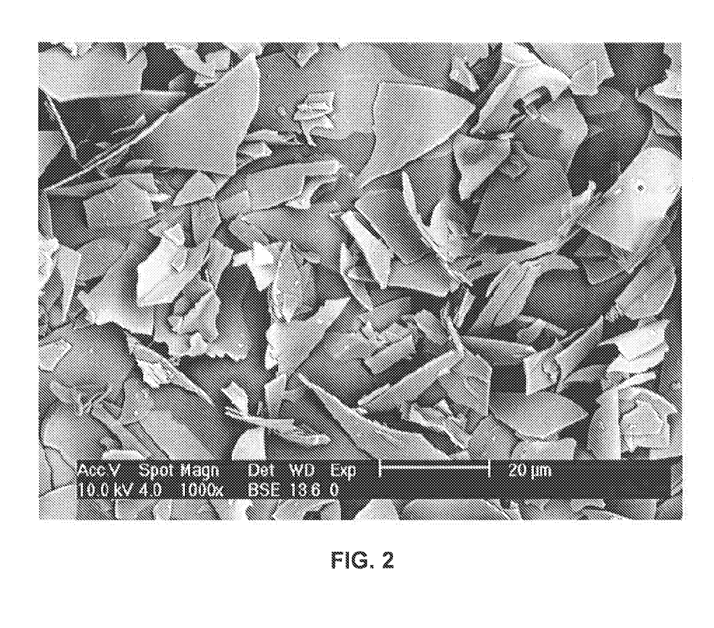

FIG. 1 shows an example of a commercially available expanded perlite (Harborlite® 2000) with large particles of natural glass. As can be seen in FIG. 1, perlite particles having a size greater than about 50 μm tend to be generally three-dimensional, multi-angular particles. In contrast, as shown in FIG. 2 fine perlite particles from the fine fraction of the classified product having a size less than about 50 μm tend to be generally two-dimensional and relatively more platy than the larger particles. Thus, it is believed that perlite particles (e.g., expanded perlite particles) having a size greater than about 50 μm tend to be more abrasive than particles having a smaller size. Further, it is also believed that smaller, platy particles tend to break down to even smaller particles more easily during, for example, a cleaning process.

According to some exemplary embodiments, perlite, for example, commercially-available perlites such as expanded perlite, may be milled and classified, such that the milled and classified perlite has a top particle size (d₉₀) less than 50 μm. For example, an un-classified, expanded perlite having a top particle size (d₉₀) of 112 μm, a median particle size (d₅₀) of 60 μm, and a (d₉₀) particle size of 22 μm may be milled and/or classified according to methods known to those skilled in art to obtain perlite having a top particle size (d₉₀) less than 50 μm. (A particle size designated “top particle size (d₉₀)” is defined as the size for which 90 percent of the volume of the particles is smaller than the indicated size. A particle size designated “median particle size (d₅₀)” is defined as the size for which 50 percent of the volume of the particles is smaller than the indicated size. A particle size designated “(d₁₀)” is defined as the size for which 10 percent of the volume of the particles is smaller than the indicated size.) For example, the milled and/or classified perlite may have a top particle size (d₅₀) less than 45 μm, such as, for example, a top particle size (d₉₀) less than 40 μm or less than 30 μm. According to some embodiments, the perlite may have a top particle size (d₅₀) ranging from 20 μm to 40 μm, such as, for example, from 25 μm to 35 μm.

According to some exemplary embodiments, the milled and/or classified perlite (e.g., expanded perlite) may have a median particle size (d₅₀) less than 30 μm. For example, the perlite may have a median particle size (d₅₀) less than 25 μm, such as, for example, a median particle size (d₅₀) less than 20 μm. Some embodiments have a median particle size (d₅₀) ranging from 5 μm to 25 μm, such as, for example, from 10 μm to 20 μm.

FIG. 2 shows an example of an expanded perlite that has been milled and/or classified in the exemplary manner described above, and it shows the relatively two-dimensional and platy nature of the milled and/or classified perlite relative to the coarse perlite shown in FIG. 1. As explained in more detail herein, compositions for cleaning teeth that include perlite with these exemplary particle size characteristics may result in compositions that result in effective cleaning of the teeth without adversely increasing the abrasiveness of the composition. Without wishing to be bound by theory, it is believed that this may result from the relatively smaller perlite particles having a relatively platy characteristic that increases the area of contact with the tooth relative to the point-like contact of the three-dimensional and angular nature of relatively larger perlite particles.

According to some embodiments, to add antimicrobial function to the perlite toothpaste abrasives, a biocidal metal compound (e.g., a nano-biocidal metal compound) may be precipitated and attached to the perlite surface to form a biocidal composition. According to some embodiments, the biocidal compositions may include at least one of zinc, copper, silver, and/or any other metal compounds known to those skilled in the art as having biocidal properties. For example, the biocidal metal compound may include zinc oxide, for example, a nano-sized zinc oxide (“nano zinc oxide”). According to some embodiments, the biocidal metal compound may range from 1 percent to 30 percent by weight of the biocidal composition. For example, the biocidal metal compound may range from 1 percent to 20 percent by weight of the biocidal composition, from 1 percent to 10 percent by weight of the biocidal composition, or from 1 percent to 5 percent by weight of the biocidal composition. According to some embodiments, the nano-biocidal particles precipitated and attached to the perlite surface may range from 1 nanometer (nm) to 250 nm. Without wishing to be bound by theory, it is believed that addition of the biocidal metal compound (e.g., coating the perlite with biocidal metal compound) may result in providing an agent for reducing or killing germs and/or bacteria associated with oral hygiene (e.g., Streptococcus mutans).

Some embodiments of biocidal compositions (e.g., boicidal abrasive compositions for cleaning teeth) include perlite in an amount ranging from, for example, 0.1 percent to 20 percent by weight of the weight of the biocidal composition, for example, from 0.1 percent to 15 percent by weight. According to some embodiments, perlite may be present in an amount ranging from, for example, 0.1 percent to 10 percent by weight of the biocidal composition, or, for example, from 0.1 percent to 3 percent by weight, for example, from 1 percent to 2 percent by weight.

According to some embodiments, the biocidal composition may also be incorporated into non-teeth-cleaning personal care products, such as, for example, deodorants and/or antiperspirants. For example, in the cosmetics field, it is well known to use, in topical application, antiperspirant products containing substances that have the effect of limiting or even suppressing the flow of sweat. These products are generally available in the form of roll-ons, sticks, aerosols, or sprays. Such products may contain, for example, perlite and one or more biocidal metal compounds, such as, for example, zinc salts, such as, zinc salicylate, zinc gluconate, zinc pidolate, zinc sulphate, zinc chloride, zinc lactate, zinc phenolsulphonate, zinc ricinoleate, sodium bicarbonate, salicylic acid and its derivatives, such as 5-n-octanoylsalicylic acid, silver zeolites, or zeolites without silver, and alum. Other biocidal metal compounds are contemplated.

According to some other embodiments, the biocidal composition may also be incorporated into sunscreens or other personal care products in order to provide protection from ultraviolet radiation. In one embodiment, the sunscreen can be in a form such as lotions, sprays, gels or other topical products that absorb or reflect some of the sun's ultraviolet (UV) radiation on the skin exposed to sunlight and thus help protect against sunburn.

In a further embodiment of the present invention, the sun care composition may further comprise one or more components selected from the group of emollients, emulsifiers, hydrants, thickeners and/or surfactants, and may be present in the form of a cream, or an ointment, or a lotion, or in a sprayable form. Such compositions have been found to have improved organoleptic properties while maintaining good SPF.

According to one embodiment of the present invention, the sun care composition may have an SPF of 15.0 or more. As used herein, “SPF” is the Sun Protection Factor, determined by in vitro measurements based on transmittance according to ISO 24445 (pre-irradiation and spectrophotometer).

According to some embodiments, a biocidal composition the perlite includes expanded perlite. The perlite may be milled and/or classified, expanded perlite. According to some embodiments, the perlite is unexpanded perlite.

Exemplary embodiments of biocidal compositions (e.g., boicidal abrasive compositions) include perlite and exhibit an RDA value less than 220. For example, some embodiments of biocidal compositions include perlite and exhibit an RDA value less than 200, for example, less than 180. Some exemplary embodiments of biocidal compositions include perlite and exhibit a PCR value of at least 110. For example, some embodiments include perlite and exhibit a PCR value of at least 120. Exemplary embodiments of biocidal compositions may include perlite and exhibit a tooth polish potential value of at least 30. For example, some embodiments may include perlite and exhibit a tooth polish potential value of at least 35.

Some embodiments of biocidal compositions are dentifrice compositions. According to some embodiments, the dentifrice composition is toothpaste, in particular, a dentifrice composition including a toothpaste base. For example, the toothpaste base may include at least one ingredient chosen from binders, such as thickening agents and/or gelling agents, humectants, foaming agents such as detergents, and polishing agents. The toothpaste base may also contain at least one additional ingredient chosen from, for example, water, preservative agents, flavoring agents, sweeteners, and fluoride containing compounds. It will be readily apparent to the skilled artisan that the components and their relative amounts in the toothpaste base may be modified to achieve the desired toothpaste product.

The toothpaste base according to some embodiments may contain at least one binder, such as thickeners, which may also be referred to as gelling agents. Any art-recognized gelling or thickening agent may be used. Thickening or gelling agents may be selected from natural, synthetic, and gum-like materials, including, but not limited to, carboxyl methyl cellulose, carrageenin, xantham gum, and bentonite. The at least one thickening or gelling agent may be present in the toothpaste base in an amount ranging from, for example, about 0.1 percent to about 5 percent by weight, for example, from about 0.1 percent to about 3 percent by weight. According to some embodiments, the at least one thickening or gelling agent is present in the toothpaste base in an amount ranging from, for example, about 0.5 percent to about 1.5 percent by weight.

According to some embodiments, the toothpaste base may also contain at least one ingredient chosen from detergents and surfactants. Suitable non-limiting examples of appropriate detergents for use in the toothpaste base include anionic surfactants, such as sodium alkylsulfates, sodium laurylsulfate, sodium myristylsulfate and sulfosuccinic acid surfactants; dialkyl sodium sulfosuccinate; non-anionic surfactants; and amphoteric surfactants. The at least one ingredient chosen from detergents and surfactants may be present in the toothpaste base in an amount ranging from, for example, about 0.1 percent to about 10 percent by weight, for example, from about 0.1 percent to about 5 percent by weight, and further, for example, from about 0.5 percent to about 3 percent by weight.

According to some embodiments, the toothpaste base may also contain at least one humectant, such as, for example, humectants chosen from glycerin, sorbitol, propylene glycols, polyethylene glycols, and mixtures thereof. The at least one humectant may be present in the toothpaste base in an amount ranging from, for example, about 10 percent to about 90 percent by weight, for example, from about 20 percent to about 80 percent by weight. According to some embodiments, the at least one humectant may be present in an amount ranging from about 30 percent to about 70 percent by weight.

Some embodiments of toothpaste base may contain at least one coloring or whitening agent. Any art-recognized coloring or whitening agent may be used. Coloring and whitening agents may include, for example, titanium dioxide. Coloring or whitening agents may be present in the toothpaste base in an amount ranging from about 0.1 percent to about 5 percent by weight, for example, ranging from about 0.1 percent to about 3 percent by weight, or, for example, ranging from about 0.1 percent to about 1 percent by weight.

The toothpaste base according to some embodiments may contain at least one preservative. Any art-recognized preservative may be used. For example, preservatives may be selected from sodium benzoate and methyl paraben. Preservatives may be present in the toothpaste base in an amount ranging from, for example, about 0.1 percent to about 3 percent, by weight, for example, ranging from about 0.1 percent to about 1 percent by weight, and further, for example, from about 0.1 percent to about 0.5 percent by weight. The toothpaste base may further contain at least one additional ingredient chosen from therapeutic ingredients and preventatives such as water-insoluble non-cationic antibacterial agents, for example, triclosan, and cationic antibacterial agents.

The toothpaste base may also contain at least one foaming agent. Any art-recognized foaming agent may be used, and appropriate foaming agents will be readily apparent to the skilled artisan. Further, the toothpaste base may contain at least one flavoring agent. Any art-recognized flavoring agent may be used, and appropriate flavoring agents will be readily apparent to the skilled artisan. For example, flavoring agents may be chosen from oils of spearmint, peppermint, wintergreen, sassafras, clove, sage, eucalyptus, cinnamon, lemon, orange, and methyl salicylate.

The toothpaste base may contain at least one sweetener. Any art-recognized sweetener may be used, and appropriate sweeteners will be readily apparent to the skilled artisan. For example, sweeteners may be chosen from at least one of sucrose, lactose, maltose, xylitol, sodium cyclamate, perillartine, aspartyl phenyl alanine methyl ester, and saccharine.

The toothpaste base may contain fluoride, such as, any compatible composition that will dissociate and release fluorine-containing ions in water. Fluoride compositions may be chosen from one or more of sodium fluoride, stannous fluoride, sodium monofluorophosphate, potassium fluoride, potassium stannous fluoride, sodium fluorostannate, stannous chlorofluoride, and amine fluoride. Fluorides may be present in the toothpaste base in an amount ranging from about, for example, 0.1 percent to about 3 percent, by weight, for example, from about 0.1 percent to about 1 percent by weight, and further, for example, from about 0.2 percent to about 0.8 percent by weight.

Compositions (e.g., boicidal abrasive compositions) according to some embodiments may also include abrasive materials chosen from any fluoride compatible abrasive material. Suitable non-limiting examples of abrasive materials that may be used may be chosen from, for example, natural glass, silica, alumina, aluminosilicate, dicalcium phosphate, sodium bicarbonate, sodium metaphosphate, potassium metaphosphate, tricalcium phosphate, calcium pyrophosphate, calcium carbonate, and bentonite. According to some embodiments, abrasives may be present in an amount ranging from about 4 percent to about 25 percent by weight, relative to the total weight of the biocidal abrasive composition.

According to some embodiments, a method of making a biocidal composition (e.g., a boicidal abrasive composition) may include providing perlite and contacting the perlite with a biocidal metal compound to form biocidal metal-treated perlite. Providing the perlite may include providing perlite having a top particle size (d₉₀) less than 50 μm and a median particle size (d₅₀) less than 30 μm. The perlite may range from 0.1 percent to 20 percent by weight of the composition. According to some embodiments, a biocidal metal compound precipitated and attached to the perlite surface may be achieved, for example, by contacting the perlite with a biocidal metal compound, which may include contacting the perlite with a metal compound including at least one of zinc, copper, and silver. For example, contacting the perlite with a biocidal metal compound may include contacting the perlite with a metal compound including zinc oxide (e.g., nano-zinc oxide) using a binder. According to some embodiments, the method may further include precipitating and attaching the biocidal metal compound on the perlite surface. According to some embodiments, the perlite may be coated with biocidal metal compound.

### EXAMPLES

### Preparation of Examples 1-3

Three examples of natural glass were prepared and tested according to the following description. Examples 1-3 were prepared using a pilot scale Alpine™ 200 ATP classifier (marketed by Hosokawa Alpine Aktiengesellchaft of Augsburg, Germany). Other classifiers known to those skilled in the art may be used. The classifier used generally includes a horizontally mounted high speed classifying wheel and a classifying air outlet. Classifying air injected into the machine base flows inwards through the classifying wheel and discharges the fine particles, and coarse particles rejected by the classifying wheel are ejected from the classifier through a coarse material outlet. By adjusting operating parameters of the classifier, such as, for example, classifier wheel speed and air flow pressure, a material having the desired characteristics may be achieved.

Examples 1-3 were obtained from a commercially-available milled, expanded perlite product, Harborlite® 2000, which was used as the feed material for the exemplary classifier described above. The feed material had a median particle size (d₅₀) of 60 μm, and a particle size distribution (PSD) from 22 μm (d₁₀) to 112 μm (d₉₀).

The particle size distribution of samples was determined in accordance with the phenomenon of scattered light from a laser beam projected through a stream of particles. The amount and direction of light scattered by the particles is measured by an optical detector array and then analyzed by a microcomputer, which calculates the size distribution of the particles in the sample stream. For example, the particle size data may be obtained on a Leeds and Northrup Microtrac ×100 laser particle size analyzer (marketed by Leeds and Northrup of North Wales, Pennsylvania). This instrument is capable of determining particle size distribution over a particle size range from 0.12 μm to 704 μm.

The color of the perlite was determined using Hunter scale “L,” “a,” and/or “b” color data collected on a Spectro/plus Spectrophotometer (Color and Appearance Technology, Inc., Princeton, N.J.). The L-value indicates the level of lightness or darkness, the a-value indicates the level of redness or greenness, and the b-value indicates the level of yellowness or blueness. Blue light brightness was calculated from the L-, a-, and b-value data. A krypton-filled incandescent lamp was used as the light source. The instrument was calibrated according to the manufacturer's instructions using a highly polished black glass standard and a factory-calibrated white opal glass standard.

Operating parameters such as classifier rotor speed and air flow pressure were adjusted to the values shown in Table 1 below to achieve Examples 1-3.

TABLE 1

| | | | Primary | Secondary | |
|---|---|---|---|---|---|
| | Rotor Speed | Feed rate | Air | Air | Yield |
| Example | (rpm) | (kg/hr) | (m³/h) | (m³/h) | (%) |
| Example 1 | 5100 | 210 | 410 | 490 | 52 |
| Example 1 | 4700 | 201 | 420 | 500 | 860 |
| Example 3 | 3700 | 211 | 430 | 500 | 72 |

The particle size distribution and color characteristics of Examples 1-3 are shown in Table 2 below.

TABLE 2

| | | | | | | | Blue Light |
|---|---|---|---|---|---|---|---|
| Example | d₁₀ | d₅₀ | d₉₀ | L | a | B | Brightness |
| Example 1 | 7.53 | 18.21 | 34.36 | 91.76 | −0.11 | 1.58 | 82.13 |
| Example 2 | 8.46 | 20.47 | 38.70 | 91.47 | −0.07 | 1.61 | 81.56 |
| Example 3 | 12.71 | 29.69 | 57.22 | 91.26 | −0.11 | 1.86 | 80.85 |

### Testing of Examples

1. Relative Dentin Abrasion Test on Dentifrices

The RDA value indicates the relative abrasion level of dentifrices. The RDA testing procedure used was the American Dental Association (ADA)-recommended procedure for determining dentifrice abrasivity. Dentin specimens were placed in a neutron flux under the controlled conditions outlined by the ADA. The specimens were then mounted in methylmethacrylate so they would fit in a V-8 cross-brushing machine. The specimens were brushed for a 1,500 stroke, precondition run using a slurry consisting of 10 grams of ADA reference material in 50 milliliters of a 0.5% carboxymethylcellulose (CMC) glycerine solution. The brushes used were those specified by the ADA, and brush tension was 150 grams.

Following a precondition run, the RDA test was performed using the above parameters (150 grams and 1,500 strokes) in a sandwich design in which each test material slurry (25 grams/40 milliliters of water) was flanked by the reference material slurries (10 grams/50 milliliters 0.5% CMC).

Samples of 1 milliliter were taken, weighed (0.01 grams) and added to 5 milliliters of scintillation cocktail. The samples were mixed well and immediately placed on the scintillation counter for radiation detection. Following counting, the net counter per minute (CPM) values were divided by the weight of the sample to calculate a net CPM/gram of slurry. The net CPM/gram of the pre- and post-ADA reference material for each test slurry was then calculated and averaged to use in the calculation for RDA for the test material. The ADA material was assigned a value of 100, and its ratio to the test material was calculated. The results of the RDA test are reported in Table 3 below. It is noteworthy to mention these RDA tests were not based on an actual toothpaste formulation, and the perlite loading percentage is significantly higher than would be used in a conventional toothpaste formulation. These test results provide relative RDA comparison between different perlite products with different top particle sizes.

2. Stained Pellicle Removal—Pellicle Cleaning Ratio Test

The PCR value is an indication of the ability of dentifrices to remove stained pellicle (i.e., an indication of the cleaning ability of dentifrice formulations). Previous studies (J. Dent. Res., 61:1236, 1982) have indicated that the results of this test with dentifrice slurries compare favorably with those obtained in controlled clinical trials. Thus, the results of this test using dentifrice slurries may be considered to predict clinical findings with a reasonable degree of confidence.

During the PCR test, bovine, permanent, central incisors were cut to obtain enamel specimens measuring approximately 10 millimeters square. The enamel specimens were then embedded in an autopolymerizing methacrylate resin, so that only the enamel surfaces were exposed. The enamel surfaces were then smoothed and polished on a lapidary wheel and lightly etched to expedite stain accumulation and adherence. The specimens were then placed on a rotating rod in a 37° C. incubator, alternately exposing them to air and to a staining broth solution consisting of trypticase soy broth, tea, coffee, mucin, FeCl₃, and Sarcina lutea (bacteria). The staining broth was changed, and the specimens were rinsed daily for approximately seven days. After seven days, a darkly-stained pellicle film was apparent on the enamel surfaces. The specimens were then rinsed, allowed to air dry, and refrigerated until use.

For purposes of the PCR test, all dentifrice examples were tested using specimens prepared at the same time. The amount of in vitro stain was graded photometrically (i.e., via a Minolta C221, colorimeter) using only the L value of the LAB scale. The area of the specimens scored was a ¼ inch diameter circle in the center of the enamel specimen. Specimens with scores between 25 and 42 (with 25 being more darkly stained) were used. On the basis of these scores, the specimens were divided into groups of 16 specimens each, with each group having the same average baseline score.

The specimens were then mounted on a mechanical V-8 cross-brushing machine equipped with soft nylon-filament (Oral-B™ 40) toothbrushes. Tension on the enamel surface was adjusted to 150 grams. The dentifrice samples were used as slurries prepared by mixing 25 grams of dentifrice with 40 milliliters of deionized water. The ADA abrasion reference material (Ca₂P₂O₇) was prepared by mixing 10 grams of the reference material in 50 milliliters of a 0.5% CMC solution. The specimens were thereafter brushed for 800 strokes (i.e., for 4% minutes). To minimize mechanical variables, one specimen per group was brushed on each of the eight brushing heads. Fresh slurries were made after being used to brush four specimens. Following brushing, specimens were rinsed, blotted dry, and scored again for stain, as previously described.

The difference between the pre- and post-brushing stain scores was determined, and the mean and standard error were calculated for the reference group. The cleaning ratio for the reference material group was assigned a value of 100. The mean decrement of the reference group was divided into 100 to obtain a constant value to multiply by each individual test decrement within the study. The individual cleaning ratio of each specimen was thereafter calculated by multiplying the decrement by the constant. The results of the PCR test are summarized in the Table 3 below.

TABLE 3

| | | RDA | PCR |
|---|---|---|---|
| | | (Relative Dentin | (Pellicle Cleaning |
| Sample ID | d₉₀ | Abrasion) | Ratio) |
| Example 1 | 34.36 | 172.12 ± 2.93 | 121.19 ± 6.76 |
| Example 2 | 38.70 | 200.54 ± 2.91 | 126.66 ± 7.47 |
| Example 3 | 57.22 | 261.55 ± 9.02 | 129.40 ± 8.29 |

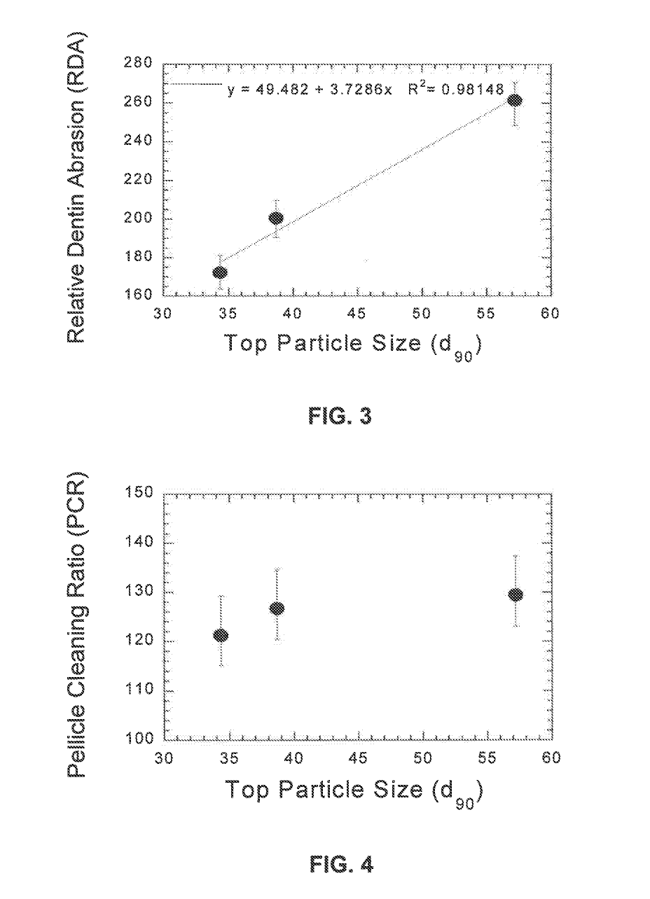

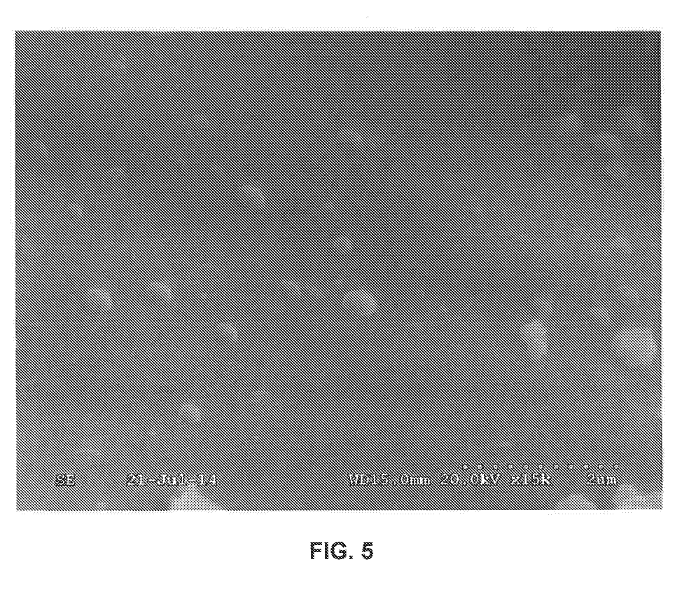

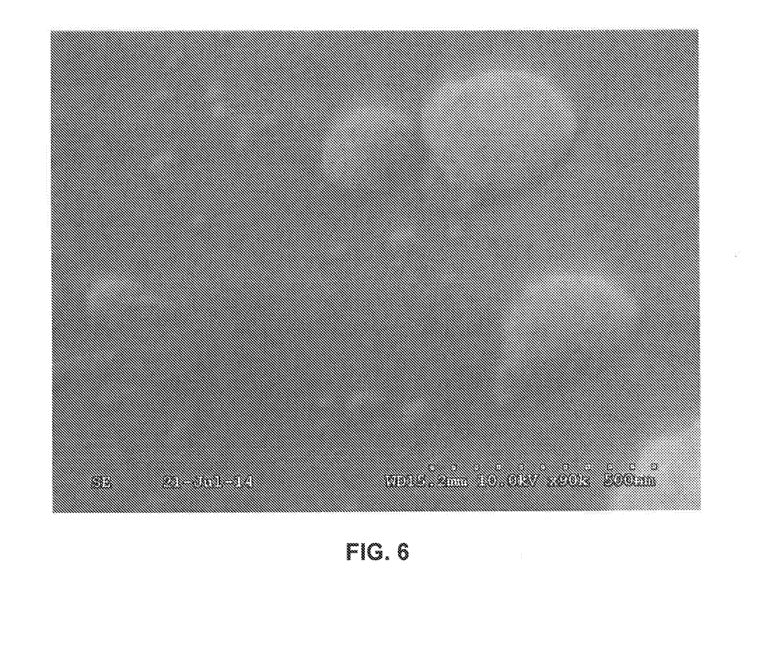

Table 3 and FIG. 3 show that abrasion decreases significantly with decreasing top particle size (d₉₀). For example, about a 30% reduction in abrasion can be achieved when top particle size (d₉₀) is less than 40 μm. As shown in FIG. 1, the perlite particles larger than 50 μm are generally three-dimensional and multi-angular in nature. Such particles tend to be more abrasive as compared to perlite particles that are less than 50 μm, which are generally two-dimensional and platy in nature. It is believed that platy particles tend easily break down during a cleaning process, which reduces abrasion. Table 3 also shows that the identified commercial baghouse perlite products have a top particle size (d₉₀) ranging from 57 μm to above 72 μm. It is believed that when particles 50 μm and larger are removed, abrasion can be significantly reduced.

Table 3 and FIG. 3 show that even with the smaller top particle size, Examples 1-3 are still effective in cleaning teeth. It is believed that this may be due to a greater contact area obtained with the relatively more platy surface of the smaller particles relative to the point-type contact with larger particles, which are relatively more three-dimensional and angular in character. Thus, compositions for cleaning teeth including perlite having a smaller top particle size provide effective cleaning and reduced erosion of the teeth.

3. Tooth Polishing

The tooth enamel polishing potential was measured using the tooth enamel surface polishing test method based on the reference. Bovine permanent, central incisors were cut to obtain labial enamel specimens measuring approximately 10×10 mm². The enamel specimens were embedded in an autopolymerizing methacrylate resin so that only the enamel surfaces were exposed. The enamel surfaces were smoothed and polished on a lapidary wheel. The bovine samples were prescreened by prophying them with a water slurry of LPA-3T abrasive (an aluminum oxide optical finishing powder) to a high luster. The samples were scored for surface reflectance using a beam reflectometer. Specimens were placed under the light source, and the entire enamel labial surface was scanned, observing the highest reading (the highest polished area). Based on a black onyx reference block being set on a reflectance value of 6.0, a polish score of at least 7.0 was achieved before any specimen was accepted for use in the study. This procedure was used to confirm the specimens' ability to achieve a high polish. The specimens were then etched by decalcifying them in 1% HCL (v/v) for 2 minutes to provide a dull surface to initiate the study. The subsequent reflectometer baseline reading was about 2.0. The specimens were also scored using the Novo-Curve Glossmeter. The specimens were scored with the glossmeter and then rotated 180° and scored again. The average of the two scores was used to calculate the glossmeter data.

Following the baseline (etched) scoring, the specimens were placed on a cross-brushing machine. The specimens were brushed for 4,500 strokes with the appropriate dentifrice slurry (25 grams of dentifrice in 40 grams of deionized water) and a medium brush (Oral B-40) at 150 g tension. The specimens were removed from the brushing machine, and rinsed and scored with the glossmeter once again for polish. Etching dulled the specimens again, and the entire procedure was repeated additional times, so that each product was assayed on each tooth set. The treatment design was a modified Latin Square design, so that no treatment followed another treatment consistently. The difference between the baseline score and the post-brushing score was calculated for each specimen as the polish increment.

Whitening toothpaste containing 2% of Example 1 shows slightly improved tooth cleaning effect relative to the same whitening toothpaste containing only high-cleaning silica as indicated by the PCR results in Table 4. RDA test results also shown in Table 4 indicate that the example perlite toothpaste is also less abrasive to teeth than the high-cleaning silica toothpaste. Compared to high-cleaning silica, the optimized perlite toothpaste abrasive significantly improves tooth polishing by about 50%, as shown in Table 4.

TABLE 4

| | PCR | RDA | |
|---|---|---|---|
| | (Pellicle | (Relative | Polish |
| | Cleaning | Dentin | Change |
| Sample ID | Ratio) | Abrasion) | (Δ Polish) |
| Whitening toothpaste | 113.32 ± 2.51 | 195.69 ± 3.04 | 37.24 ± 3.29 |
| containing 2% Example | | | |
| 1 and 18% high cleaning | | | |
| silica (Zeodent ® 105) | | | |
| Whitening toothpaste | 109.13 ± 2.93 | 211.29 ± 5.09 | 24.56 ± 4.23 |
| containing 20% high | | | |
| cleaning silica | | | |
| (Zeodent ® 105) | | | |

### Preparation of Examples 4-6

A desired amount of zinc acetate (Table 5) was dissolved in the water at room temperature. The pH of the zinc acetate solution was adjusted to around 6.3 by adding NaOH (Table 5). The zinc acetate solution was then slowly added to 40 g of abrasive composition including perlite (e.g., Example 1, PerlClean)) in a Hobart food mixer. After mixing for 15 minutes, the mixture was brushed through a 14 mesh screen with a 1.40 mm opening. After drying in a 150° C. oven overnight, the material was brushed through a 20 mesh (0.85 mm opening) screen.

TABLE 5

| | PerlClean | Zinc acetate | Water | NaOH | |
|---|---|---|---|---|---|
| Sample ID | (g) | (g) | (g) | (g) | pH |
| Example 4 | 40 | 2 | 20 | 1.00 | 6.34 |
| Example 5 | 40 | 4 | 20 | 6.15 | 6.33 |
| Example 6 | 40 | 6 | 20 | 11.5 | 6.33 |

Previous studies show that although perlite particles may be effective in tooth cleaning, the coarse perlite products such as Harborlite® 2000 may be too abrasive and may cause grooves on the tooth surface. This may be due to the extensive contact of the sharp point cutting edges of large three-dimensional angular particles (see, e.g., FIG. 1) on the tooth surface, which may result in excessive abrasion on the tooth. In contrast, fine perlite particles under 50 microns have a two-dimensional platy structure (see, e.g., FIG. 2). During the tooth brushing process, these small platy perlite particles increase the area of contact with the tooth surface relative to the sharp point-like contact of the three-dimensional and angular larger particles, and thus, may reduce tooth abrasion while maintaining the similar cleaning effect. Based on the PCR and RDA results, the finest classified perlite (Example 1) shows improved toothpaste abrasive performance with good tooth cleaning effect and significantly lower tooth abrasion. The above-noted test results in the whitening toothpaste confirm that the small platy perlite particles may slightly improve tooth cleaning and may reduce tooth abrasion as compared to the high-cleaning silica in the same toothpaste formulation.

The optimized perlite particle size and morphology may also contribute to the significant improvement in tooth polishing as compared to the high-cleaning silica in the same whitening toothpaste formulation. Although coarse particles may remove the stain on the tooth surface, they may also remove hard tooth structure and produce large scratches on the tooth surface. In contrast, the fine particles have been found to produce relatively small scratches with less damage to the tooth. By controlling the perlite top particle size through air classification, the perlite particle size may be reduced sufficiently, so the scratches become extremely fine and may disappear entirely, to produce a smooth shiny surface layer. Visualization studies have shown that the perlite particles remain in a relatively flat orientation under toothbrush tips. The small platy perlite particles scratch the teeth with edges rather than sharp points as with granular silica. As a result, the scratches by the platy perlite may be shorter and shallower. Under the work-load pressure applied by the tooth brush, the perlite plates may also break down to smaller plates, and the initial sharp edges of the plates may quickly become more rounded to produce even finer scratches for better polishing.

### Ant-Microbial Testing

A direct bacterial killing experiment was done using spiral plating to measure the viability of the Streptococcus mutans UA159 (ATCC 700610) (S. mutans). Streptococcus mutans is facultatively anaerobic, gram-positive coccus-shaped bacterium commonly found in the human oral cavity and is a significant contributor to tooth decay.

All of Examples 4-6 were measured blindly without any knowledge of their composition. This was accomplished by pipetting 1 ml of the diluted sample powder particles (100 mg/ml) into 3 ml of Tryptic Soy broth containing 1% sucrose (TSBS) and 20 ul of an overnight culture of S. mutans. The inoculated suspensions were incubated for 24 hours at 37° C. in 5% CO₂, diluted 1:10 and 1:1000, and spiral plated onto blood agar plates. The colonies on the plates were counted in a Colony Forming Unit (CFU) after 24 hours of incubation. Regarding controls, 0.12% chlorhexidine (CHX) was used a positive control, and sterile water was used as a negative control.

### Anti-Microbial Test Results

The results of the anti-microbial tests are shown in Table 6 below. As shown in Table 6, the positive CHX controls significantly inhibited the S. mutans in the viability assay. The sterile water did not inhibit any bacteria. The uncoated sample supported the growth of S. mutans, the most of any of the samples. Conversely, all nano zinc oxide coated samples inhibited S. mutans. Example 6 with the highest nano zinc oxide concentration showed most inhibition. Example 6 reduced S. mutans by two orders of magnitude compared to the uncoated sample.

TABLE 6

| Sample ID | Mean (CFU/ml) |
|---|---|
| CHX control | 1.67 × 10² ± 0.047 |
| PerlClean ™ | 1.35 × 10⁷ ± 0.335 |
| Example 4 | 2.40 × 10⁶ ± 0.06 |
| Example 5 | 8.67 × 10⁶ ± 0 |
| Example 6 | 1.02 × 10⁵ ± 0 |

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A biocidal composition comprising:
perlite; and
a biocidal metal compound associated with the perlite,
wherein the perlite has a top particle size (d₉₀) less than 50 μm and a median particle size (d₅₀) less than 30 μm, and
wherein the perlite ranges from 0.1 percent to 20 percent by weight of the composition.

2. The composition of claim 1, wherein the perlite comprises expanded perlite.

3. The composition of claim 1, wherein the perlite comprises milled and classified, expanded perlite.

4. The composition of claim 1, wherein the perlite comprises unexpanded perlite.

5. The composition of claim 1, wherein the biocidal metal compound comprises at least one of zinc, copper, and silver.

6. The composition of claim 5, wherein the biocidal metal compound comprises zinc oxide.

7. The composition of claim 5, wherein the biocidal metal compound comprises biocidal metal compound particles, and wherein the biocidal metal compound particles are attached to a surface of the perlite and have a particle size less than 250 nm.

8-16. (canceled)

17. The composition of claim 1, wherein the biocidal metal compound ranges from 1 percent to 20 percent by weight of the composition.

18-19. (canceled)

20. The composition of claim 1, wherein the perlite has a top particle size (d₉₀) less than 40 μm.

21-23. (canceled)

24. The composition of claim 1, wherein the perlite ranges from 0.1 percent to 5 percent by weight of the composition.

25. (canceled)

26. The composition of claim 1, wherein the biocidal composition exhibits an RDA value less than 200.

27-28. (canceled)

29. The composition of claim 1, wherein the biocidal composition exhibits an RDA value less than 80.

30-33. (canceled)

34. The composition of claim 1, further comprising a toothpaste base.

35-42. (canceled)

43. A dentifrice composition comprising:
perlite; and
a biocidal metal compound associated with the perlite,
wherein the perlite has a top particle size (d₉₀) less than 50 μm, a median particle size (d₅₀) less than 30 μm, and the dentifrice composition exhibits an RDA value less than 200.

44. The composition of claim 43, wherein the perlite comprises expanded perlite.

45-46. (canceled)

47. The composition of claim 43, wherein the biocidal metal compound comprises at least one of zinc, copper, and silver.

48. The composition of claim 47, wherein the biocidal metal compound comprises zinc oxide.

49. (canceled)

50. The composition of claim 43, wherein the biocidal metal compound ranges from 1 percent to 10 percent by weight of the composition.

51. (canceled)

52. The composition of claim 43, wherein the perlite has a top particle size (d₉₀) less than 40 μm.

53-64. (canceled)

65. A method of making a biocidal composition, the method comprising;
providing perlite; and
contacting the perlite with a biocidal metal compound to form biocidal metal-treated perlite,
wherein providing the perlite comprises providing perlite having a top particle size (d₉₀) less than 50 μm and a median particle size (d₅₀) less than 30 μm, and
wherein the perlite ranges from 0.1 percent to 20 percent by weight of the composition.

66-79. (canceled)
